(19) United States
(12) Patent Application Publication
(10) Pub. No.: US 2025/0320811 A1
(43) Pub. Date: Oct. 16, 2025

(54) WELL COMPLETION OPTIMIZATION

(71) Applicant: Saudi Arabian Oil Company, Dhahran (SA)

(72) Inventors: Mohd Azizi Ibrahim, Dhahran (SA); Moustafa Hamdy Dawoud, Dhahran (SA); Abdalrasheed Hasan Hashem, Dhahran (SA)

(21) Appl. No.: 18/635,332

(22) Filed: Apr. 15, 2024

(51) Int. Cl. E21B 47/09; G01V 20/00

(52) U.S. Cl. CPC E21B 47/09; G01V 20/00; E21B 2200/20

(57) ABSTRACT

Disclosed are methods, systems, and computer-readable media to perform operations including: performing a Multimineral (MM) petrophysical evaluation using a MM petrophysical model; calibrating the MM petrophysical model to core data of a well in the reservoir; performing a shaly-sand-analysis (SSA) evaluation using a SSA petrophysical model; calibrating the SSA petrophysical model to core analysis volumetric result of the reservoir; calibrating porosity (PHIT) and water saturation (SW) of the SSA petrophysical model to PHIT and SW output from the MM petrophysical model; identifying intervals of the reservoir having a volume of sand (VSD) more than a first threshold value, a permeability more than a second threshold value, and a gas saturation more than a third threshold value; calibrating the VSD, the permeability, and the gas saturation of the intervals to dynamic data of the reservoir; generating a strategic completion optimization planner plot indicating perforation zones within intervals.

### TECHNICAL FIELD

This disclosure relates generally to planning and optimizing well-completion operations, and more particularly, a Strategic Completion Optimization Planner (SCOP).

### BACKGROUND

A reservoir refers to a subsurface rock formation that contains economically recoverable hydrocarbons, such as oil and natural gas. Reservoirs can consist of various types of rocks, including sandstone, limestone, and shale.

Well completion refers to the process of preparing an oil or gas well for production after drilling has been completed. It involves a series of steps and activities to establish a pathway for hydrocarbons to flow from the reservoir to a wellbore, as well as maintaining the integrity and safety of the wellbore.

### SUMMARY

The present disclosure describes a Strategic Completion Optimization Planner (SCOP) identifying an effective perforation zone for hydrocarbon production.

The described subject matter can be implemented using a computer-implemented method; a non-transitory, computer-readable medium storing computer-readable instructions to perform the computer-implemented method; and a computer-implemented system comprising one or more computer memory devices interoperably coupled with one or more computers and having tangible, non-transitory, machine-readable media storing instructions that, when executed by the one or more computers, perform the computer-implemented method/the computer-readable instructions stored on the non-transitory, computer-readable medium.

According to one innovative aspect of the present disclosure, a computer-implemented method for identifying a perforation zone for a reservoir, including: performing, by one or more processors, a Multimineral (MM) petrophysical evaluation using a MM petrophysical model; calibrating, by the one or more processors, the MM petrophysical model to core data of a well in the reservoir; performing, by the one or more processors, a shaly-sand-analysis (SSA) evaluation using a SSA petrophysical model; calibrating, by the one or more processors, the SSA petrophysical model to core analysis volumetric result of the reservoir; calibrating, by the one or more processors, porosity (PHIT) and water saturation (SW) of the SSA petrophysical model to PHIT and SW output from the MM petrophysical model; identifying, by the one or more processors, one or more intervals of the reservoir having a volume of sand (VSD) more than a first threshold value, a permeability more than a second threshold value, and a gas saturation more than a third threshold value; calibrating, by the one or more processors, the VSD, the permeability, and the gas saturation of the one or more intervals to dynamic data of the reservoir; and generating, by the one or more processors, a strategic completion optimization planner (SCOP) plot indicating one or more perforation zones within the one or more intervals.

The innovative method can include other optional features. For example, in some implementations, generating the SCOP plot indicating the one or more perforation zones is performed in response to the VSD, the permeability, and the gas saturation matching the dynamic data at a degree more than a fourth threshold value.

In some implementations, the dynamic data comprises measurements from a Formation Testing with Sampling (FTS) tool and perforation results.

In some implementations, the method further including: generating a gross sand flag in response to the VSD of the one or more intervals being more than the first threshold value; generating a permeable layer flag in response to the permeability of the one or more intervals being more than the second threshold value; and generating a hydrocarbon flag in response to the gas saturation of the one or more intervals being more than the third threshold value.

In some implementations, the method further including: obtaining the core analysis volumetric result by deterministic description or advanced mud logging (AML) X-Ray Diffraction (XRD)/X-Ray Fluorescence (XRF).

In some implementations, the core data comprises PHIT, a permeability, and SW data of the well.

In some implementations, the SCOP plot comprises a perforation depth and a perforation thickness of each perforation zone.

According to another innovative aspect of the present disclosure, a non-transitory, computer readable, storage medium that stores instructions that, when executed by at least one processor, cause the at least one processor to perform operations including: performing a Multimineral (MM) petrophysical evaluation using a MM petrophysical model; calibrating the MM petrophysical model to core data of a well in a reservoir; performing a shaly-sand-analysis (SSA) evaluation using a SSA petrophysical model; calibrating the SSA petrophysical model to core analysis volumetric result of the reservoir; calibrating porosity (PHIT) and water saturation (SW) of the SSA petrophysical model to PHIT and SW output from the MM petrophysical model; identifying one or more intervals of the reservoir having a volume of sand (VSD) more than a first threshold value, a permeability more than a second threshold value, and a gas saturation more than a third threshold value; calibrating the VSD, the permeability, and the gas saturation of the one or more intervals to dynamic data of the reservoir; and generating a strategic completion optimization planner (SCOP) plot indicating one or more perforation zones within the one or more intervals.

The innovative medium can include other optional features. For example, in some implementations, generating the SCOP plot indicating the one or more perforation zones is performed in response to the VSD, the permeability, and the gas saturation matching the dynamic data at a degree more than a fourth threshold value.

In some implementations, the dynamic data comprises measurements from a Formation Testing with Sampling (FTS) tool and perforation results.

In some implementations, the operations further including: generating a gross sand flag in response to the VSD of the one or more intervals being more than the first threshold value; generating a permeable layer flag in response to the permeability of the one or more intervals being more than the second threshold value; and generating a hydrocarbon flag in response to the gas saturation of the one or more intervals being more than the third threshold value.

In some implementations, the operations further including: obtaining the core analysis volumetric result by deterministic description or advanced mud logging (AML) X-Ray Diffraction (XRD)/X-Ray Fluorescence (XRF).

In some implementations, the core data comprises PHIT, a permeability, and SW data of the well.

In some implementations, the SCOP plot comprises a perforation depth and a perforation thickness of each perforation zone.

According to another innovative aspect of the present disclosure, a computer-implemented system, including: one or more memory modules; one or more hardware processors communicably coupled to the one or more memory modules, the one or more hardware processors configured to execute instructions stored on the one or more memory models to perform operations including: one or more memory modules; one or more hardware processors communicably coupled to the one or more memory modules, the one or more hardware processors configured to execute instructions stored on the one or more memory models to perform operations comprising: performing a Multimineral (MM) petrophysical evaluation using a MM petrophysical model; calibrating the MM petrophysical model to core data of a well in a reservoir; performing a shaly-sand-analysis (SSA) evaluation using a SSA petrophysical model; calibrating the SSA petrophysical model to core analysis volumetric result of the reservoir; calibrating porosity (PHIT) and water saturation (SW) of the SSA petrophysical model to PHIT and SW output from the MM petrophysical model; identifying one or more intervals of the reservoir having a volume of sand (VSD) more than a first threshold value, a permeability more than a second threshold value, and a gas saturation more than a third threshold value; calibrating the VSD, the permeability, and the gas saturation of the one or more intervals to dynamic data of the reservoir; and generating a strategic completion optimization planner (SCOP) plot indicating one or more perforation zones within the one or more intervals.

The innovative medium can include other optional features. For example, in some implementations, generating the SCOP plot indicating the one or more perforation zones is performed in response to the VSD, the permeability, and the gas saturation matching the dynamic data at a degree more than a fourth threshold value.

In some implementations, the dynamic data comprises measurements from a Formation Testing with Sampling (FTS) tool and perforation results.

In some implementations, the operations further including generating a gross sand flag in response to the VSD of the one or more intervals being more than the first threshold value; generating a permeable layer flag in response to the permeability of the one or more intervals being more than the second threshold value; and generating a hydrocarbon flag in response to the gas saturation of the one or more intervals being more than the third threshold value.

In some implementations, the operations further including obtaining the core analysis volumetric result by deterministic description or advanced mud logging (AML) X-Ray Diffraction (XRD)/X-Ray Fluorescence (XRF).

In some implementations, the core data includes PHIT, a permeability, and SW data of the well.

The subject matter described in this specification can be implemented to realize one or more of the following advantages. First, the described subject matter can provide a robust approach to identify the most likely effective clastic reservoir package to be perforated for the well, thereby improving well productivity. Second, the described subject matter can reduce costs by not perforating nonproductive zones. Third, the described subject matter can precisely locate a depth of permeable zone layers that are most likely injectable where fracture ports are strategically placed. Fourth, the described subject matter can optimize the number of perforation intervals in a clastic reservoir. Fifth, the described subject matter can increase a success rate in injecting operations by understanding impact of silt on permeability in a clastic reservoir. Sixth, the described subject matter can identify vertical connected sandstone layers within a clastic reservoir and bound fluid/water zones. Seventh, the described subject matter can demonstrate variations in sand-silt composition related to a depositional environment for mapping effective reservoir distribution within a field.

The details of one or more implementations of the subject matter of this specification are set forth in the Detailed Description, the Claims, and the accompanying drawings. Other features, aspects, and advantages of the subject matter will become apparent to those of ordinary skill in the art from the Detailed Description, the Claims, and the accompanying drawings.

### BRIEF DESCRIPTION OF THE DRAWINGS

FIG. 1 illustrates a flow chart of an example process performed by a Strategic Completion Optimization Planner (SCOP) for a clastic reservoir, according to some implementations.

FIG. 2A is an example plot of Multimineral (MM) evaluation illustrating mineral components of the clastic reservoir, according to some implementations.

FIG. 2B is an example plot of Shaly Sand Analysis (SSA) evaluation illustrating sand-silt shales with variations in rock quality of the clastic reservoir, according to some implementations.

FIG. 2C is an example plot illustrating the combined MM evaluation and SSA evaluation of the clastic reservoir, according to some implementations.

FIG. 3 is an example plot illustrating two experimental perforation zones, according to some implementations.

FIG. 4 illustrates a flow chart of a process for identifying a perforation zone for the production of hydrocarbons, according to some implementations.

FIG. 5 illustrates hydrocarbon production operations that include both one or more field operations and one or more computational operations, which exchange information and control exploration for the production of hydrocarbons, according to some implementations.

FIG. 6 is a schematic illustration of an example controller (or control system) that enables a SCOP to identify a perforation zone for the production of hydrocarbons, according to some implementations.

### DETAILED DESCRIPTION

This disclosure describes a Strategic Completion Optimization Planner (SCOP) identifying an effective perforation zone for hydrocarbon production and is presented to enable any person skilled in the art to make and use the disclosed subject matter in the context of one or more particular implementations. Various modifications, alterations, and permutations of the disclosed implementations can be made and will be readily apparent to those of ordinary skill in the art, and the general principles defined can be applied to other implementations and applications, without departing from the scope of the present disclosure. In some instances, one or more technical details that are unnecessary to obtain an understanding of the described subject matter and that are within the skill of one of ordinary skill in the art may be omitted so as not to obscure one or more described implementations. The present disclosure is not intended to be limited to the described or illustrated implementations, but to be accorded the widest scope consistent with the described principles and features.

The techniques described herein can identify the most likely injectable zone within a complex multilayered clastic reservoir for perforation and production operation. The techniques combine a Shaly Sand Analysis (SSA) model that performs deterministic petrophysical analysis with a Multimineral (MM) petrophysical model that performs statistical analysis. The combined models are calibrated to dynamic data from a formation tester and sampling (FTS) and production results (e.g., production rates, pressure data, fluid composition data, etc.) to identify the best perforation zone. The MM and SSA petrophysical models are calibrated to the dynamic data within a geological setting, so as to better align with observed reservoir behaviors and properties. The dynamic data relates to well productivity, and can include, e.g., flow rate, fluid types, mobility, flowing pressure.

Some advantages of the present techniques include: (I) providing a robust approach to identify the most likely effective clastic reservoir package to be perforated for the well, thereby improving well productivity; (II) reducing costs by not perforating nonproductive zones; (III) precisely locating a depth of permeable zone layers that are most likely injectable where fracture ports are strategically placed; (IV) optimizing the number of perforation intervals in a clastic reservoir; (V) increasing a success rate in injecting operations by understanding impact of silt on permeability in a clastic reservoir; (VI) identifying vertical connected sandstone layers within a clastic reservoir and bound fluid/water zones; (VII) demonstrating variations in sand-silt composition related to a depositional environment for mapping effective reservoir distribution within a field.

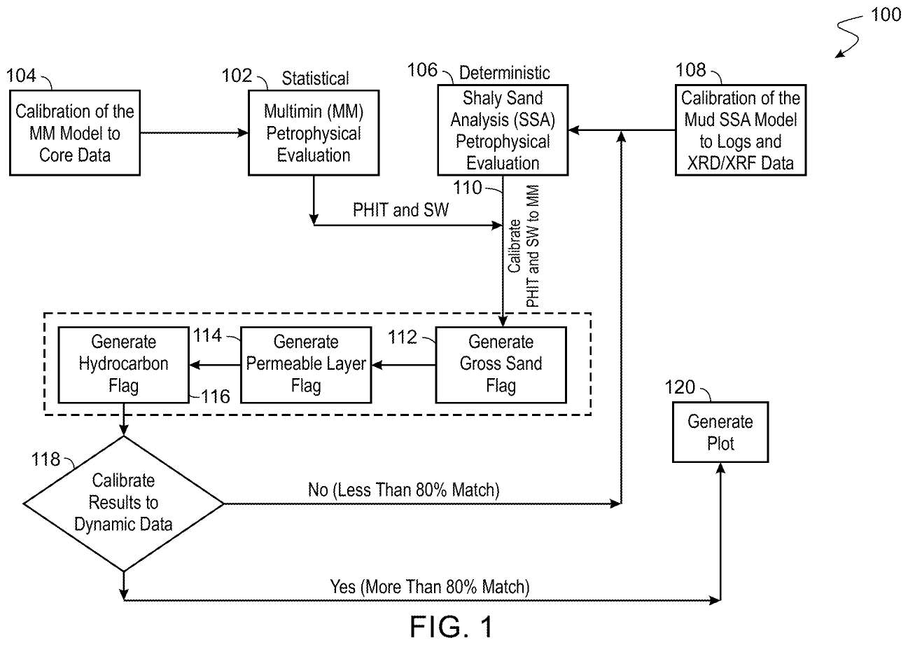

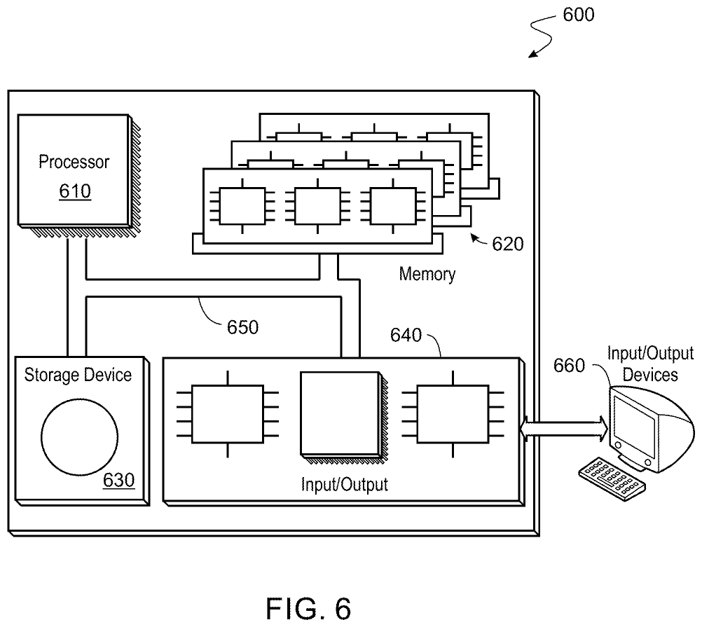

FIG. 1 illustrates a flow chart of an example process 100 performed by a SCOP for a clastic reservoir, according to some implementations. The SCOP can identify the most likely effective perforation zone for hydrocarbon production. The process 100 is described as being performed by a computing device including one or more processors or a controller, such as controller 600 of FIG. 6. The process 100 shown in FIG. 1 can be modified or reconfigured to include additional, fewer, or different steps (not shown in FIG. 1), which can be performed in the order shown or in a different order.

At 102, the controller 600 builds a MM petrophysical model for MM petrophysical evaluation. The MM petrophysical model is a tool used for analyzing mineral compositions in geological samples. MM petrophysical model can involve techniques such as X-ray diffraction (XRD) or X-ray fluorescence (XRF) analysis to determine mineralogical composition of rocks or sediments. There is no restriction to the software used for this MM evaluation. For example, GEOLOG by Paradigm, ELAN by Schlumberger, or any other customized software can be used for MM evaluation. The output of the MM petrophysical model includes interpretations of mineral components such as Quartz, Illite, Kaolinite, and Orthoclase, as well as fluid (e.g., oil or gas) information of a reservoir. These interpretations are derived from well-log data, core samples, correlation, or dynamic data from a formation tester using the MM petrophysical model.

At 104, the controller 600 calibrates the MM petrophysical model to the available core data 101, e.g., core porosity (Porosity of Hydrocarbon-Intervals in Thin Beds (PHIT)), permeability, and water saturation (SW) data. Core data refers to measurements and analyses conducted on rock core samples retrieved from the subsurface during drilling operations. A core is a rock sample taken in the form of a cylinder or a plug. The core and its plug can be analyzed for petrophysical properties. The MM petrophysical model parameters are adjusted to match the observed properties measured from core samples. Permeability is a measure of how easily fluids can flow through the rock formation. Permeability data indicates the reservoir's ability to produce fluids. Calibration of the MM petrophysical model to available PHIT, permeability, and SW data can improve the accuracy and reliability of the MM petrophysical model's predictions and enhance understanding of the reservoir's lithology, mineralogy, and fluid distribution.

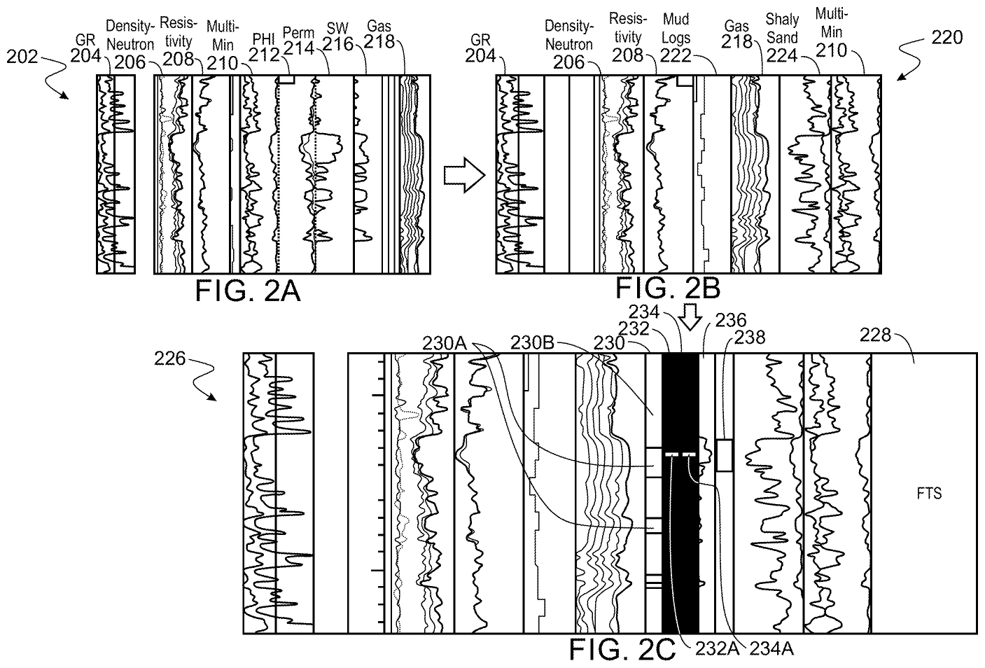

FIG. 2A is an example plot 202 of an MM evaluation illustrating mineral components of the elastic reservoir, according to some implementations. The example plot 202 includes a Gamma Ray (GR) 204, Density-Neutron (DN) 206, resistivity 208, MM 210, PHIT212, Permeability (PERM) 214, SW 216, and Gas Content (GAS) 218. GR logs 204 measure natural radiation emitted by rock formation and are used to identify lithology and correlate rock units. GR logs 204 can be used to distinguish between different rock types, including shale, sandstone, and limestone. DN logs 206 provide information about the bulk density and porosity of the rock formation. DN logs 206 are used to estimate porosity and differentiate between porous and non-porous intervals. Resistivity logs 208 measure the electrical resistivity of the formation, which is influenced by the presence of fluids and minerals. Resistivity logs 208 can be used to identify hydrocarbon-bearing zones, evaluate formation SW, and estimate formation resistivity factor. MM 210 involves quantifying the mineral composition of the reservoir rock based on well-log data or core samples. It can be used to characterize lithology, identify mineralogical variations, and assess reservoir quality. PHIT212 is a measure of the volume of pore space within the rock formation. Porosity logs are used to estimate the amount of pore space available for fluid storage, including oil, gas, and water. Permeability 214 refers to the ability of the rock to transmit fluids through its pore spaces. Permeability logs or derived permeability values are used for predicting fluid flow behavior and assessing reservoir productivity. SW 216 represents the fraction of pore space filled with formation water relative to the total pore volume. SW logs 216 are used to evaluate reservoir quality and estimate the volume of hydrocarbons in place. GAS 218 refers to the amount of gas present within the pore spaces of the reservoir rock. GAS logs 218 can assess reservoir productivity, estimate gas reserves, and optimize production strategies.

At 106, the controller 600 builds a SSA petrophysical model. SSA evaluation is a technique used to characterize sandstone reservoirs that contain significant amounts of clay minerals (shale). SSA evaluation can involve petrophysical analysis of well logs (such as GR logs, DN logs) to identify and quantify the presence of shale within sandstone reservoirs. There is no restriction to the software used for the SSA evaluation. For example, GEOLOG by Paradigm, PETROVIEW by Schlumberger, CROCKER SHALY SAND by Crocker, or any other customized software can be used for the SSA evaluation.

At 108, the controller 600 calibrates the SSA's volume of sand (VSD), volume of silt (VST), and volume of shale (VSH) to core analysis volumetric result. The core analysis volumetric result can be obtained by deterministic description or using advanced mud logging (AML) XRD and XRF. XRD and XRF are two analytical techniques used in geology and materials science for determining the mineralogical and elemental composition of solid samples, including rocks, minerals, and soils.

FIG. 2B is an example plot 220 of SSA evaluation illustrating sand-silt shales with variations in rock quality of the clastic reservoir, according to some implementations. SSA evaluates shaly sand intervals to assess their reservoir quality, mineral composition, and fluid content, which impact reservoir performance. The example plot 220 includes a GR 204, DN 206, resistivity 208, mud logs 222, GAS 218, SSA 224, and MM 210. Mud logs 222 involve analyzing drill cuttings and drilling fluids to identify lithology, hydrocarbon shows, and formation pressures encountered during drilling. Mud logs provide real-time information for correlation with other logging measurements and geological analyses. SSA 224 refers to lithological units containing a significant proportion of both sand and shale components.

At 110, the controller 600 calibrates PHIT and SW of the SSA to the MM petrophysical parameters (PHIT and SW) output from the MM petrophysical model. The MM petrophysical parameters PHIT and SW were calibrated to the core data at 104.

At 112, the controller 600 generates a gross sand flag if VSD of a rock interval is above a threshold value. For example, the threshold value of VSD can be a value between 0.70 and 0.50. The gross sand flag is a designation to indicate the presence of a predefined thickness or volume of sand within a geological formation.

At 114, the controller 600 calculates permeability using a continuous log permeability model and generates a permeable layer flag if the permeability of a rock interval is above a threshold value. For example, the threshold value of permeability can be a value of 0.1 millidarcies (mD) or higher for a sandstone reservoir, depending on the fluid and rock properties. Different types of reservoirs can have different threshold values. The continuous log permeability model relates well-log measurements (e.g., PHIT, resistivity) to permeability through empirical correlations or mathematical equations. The permeable layer flag is a designation used in reservoir characterization to identify intervals or layers within a geological formation that exhibit a predefined permeability.

At 116, the controller 600 analyzes mud log gas data and generates a hydrocarbon flag (e.g., a gas flag) indicating a productive gas zone if a gas saturation or a gas-to-oil ratio of an interval is above a threshold value. For example, the threshold value can be around 50% for a gas reservoir (the gas accounts for around 50% of the total mud gas measurement). The hydrocarbon flag is a designation used in reservoir characterization to identify intervals or layers within a geological formation that contain hydrocarbons, such as oil or gas. Mud log gas data refers to the measurements and analysis of gases obtained from drilling mud during a drilling process in oil and gas exploration. The Mud log gas analysis is a technique used in drilling operations to monitor the composition of gases encountered while drilling a well. It involves continuously analyzing gas samples extracted from the drilling mud circulating in the wellbore. The hydrocarbon flag is generated after correlation/calibration with perforation/formation tester results. Correlation of field data refers to a process of establishing a relationship between different rock formations or geologic units in different wells or locations.

The threshold values at 112-116 are dependent on a reservoir type and a mud type. For example, at 114, the threshold value of permeability for a tight rock with a light fluid can be 0.1 mD, while the threshold value of permeability for a heavier viscous fluid can be up to 10 mD.

At 118, one or more gross sand layers indicated by the gross sand flag(s), one or more permeable layers indicated by the permeable layer flag(s), and one or more productive gas zones indicated by the hydrocarbon flag(s) are calibrated to dynamic data of the reservoir. If the results at 112-116 (e.g., VSD, permeability, gas saturation, or a gas-to-oil ratio) match 80% or more to dynamic data, the controller 600 proceeds to 120. If the results at 112-116 match less than 80% of the dynamic data, the controller 600 reverts to 106. The dynamic data encompasses measurements and observations obtained during the production and operation of oil and gas reservoirs. The dynamic data can include measurements using a Formation Testing with Sampling (FTS) tool, as well as data related to perforation results. FTS involves the collection of fluid samples from the reservoir using downhole tools. The sampled fluids are analyzed to determine properties for reservoir characterization and production planning, such as composition, pressure, and fluid phase behavior. An effective or injectable zone can be correlative with the mobility/permeability measured by FTS. FTS can also confirm the fluid for a particular zone in the reservoir. Perforation results include perforation operation data, such as perforation depth, perforation diameter, perforation density (the number of perforations per unit length), and the condition of rock formation surrounding the perforations.

FIG. 2C is an example plot 226 illustrating combined MM evaluation and SSA evaluation of the clastic reservoir, according to some implementations. The example plot 226 includes FTS data 228, mud log gas analysis 230, effective sand data 232, perforation depth data 234, and log-calculated permeability 236 that is calibrated to the core permeability measurement.

FTS data 228 includes composition, pressure, temperature, and fluid behavior of the reservoir.

Mud log gas analysis 230 involves measuring the concentration of various gases in drilling mud, particularly hydrocarbon gases such as methane (CH4), ethane (C2H6), propane (C3H8), and butane (C4H10). These gases can originate from hydrocarbon reservoirs, indicating the presence of oil or gas formations. The mud log gas analysis 230 outputs indicators of gas or hydrocarbon 230A and indicators of water 230B. The mud log gas analysis 230 is based on the total hot mud gas for the well cutoff in parts per million (ppm).

The effective sand zone refers to a portion of the reservoir that is considered to be productive or capable of producing hydrocarbons. Effective sand data 232 indicates the quality and thickness of the reservoir interval that contributes to production. The effective sand data 232 includes a thickness of an effective sand 232A.

Perforation depth data 234 indicates depth intervals in the wellbore where perforation holes are made in the casing or liner. These perforation holes allow hydrocarbons to flow from the reservoir into the wellbore, facilitating production. The perforation depth data 234 includes a thickness of an injectable zone 234A.

A packer 238 for fracking is placed at a depth corresponding to a depth of the injectable zone. A packer 238 is a mechanical device used to create a seal between different sections of the wellbore or between the wellbore and the casing or tubing. The packer 238 can isolate a specific zone in the well, control production or injection flow, and prevent fluid migration between different rock formations or zones.

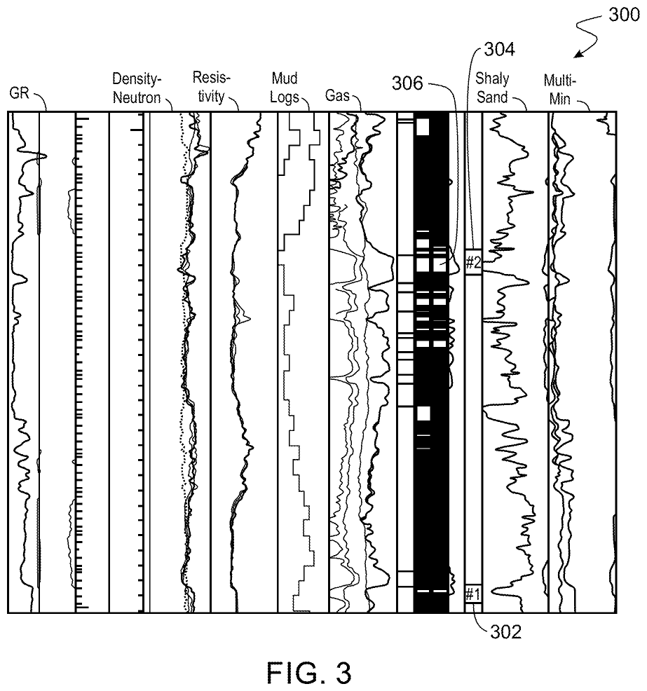

At 120, the controller 600 generates an example plot (e.g., plot 300 of FIG. 3) that visualizes perforation zones, e.g., a plot that illustrates the distribution and characteristics of perforation zones in a wellbore. Criteria for selecting perforation zones include reservoir properties, rock formation evaluation data, geological analysis, fluid composition, pressure and temperature, production history, completion objectives, wellbore stability, economic factors, etc. The reservoir properties are determined based on field data and production history in an area where a wellbore is located.

Field data refers to the information collected directly from an oilfield or exploration site. The field data includes various types of measurements, observations, and tests conducted on-site to characterize the geological, geophysical, and engineering properties of the subsurface reservoir. The field data can include geological surveys, seismic data, well logs, well test results, production data, surface and subsurface measurements, and other relevant information obtained during field operations.

The production history refers to a record of oil or gas production from a reservoir over a certain period of time. The production history includes data on the volume of hydrocarbons extracted, production rates, well performance, reservoir pressure, and other relevant parameters.

Referring to FIG. 3, FIG. 3 is an example plot 300 illustrating two experimental perforation zones, according to some implementations. The example plot 300 illustrates two experimental perforation zones 302 and 304. In some examples, a hydraulic fracturing fluid (frac mud) is injected into a reservoir zone through perforation zone 302. The perforation zone 302 is determined without using the example process 100. The reservoir zone has a low gas flow rate that does not meet production requirements. The reservoir zone is a low permeability zone with negligible wellhead pressure, and siltstone layers above and below a sandstone layer in the reservoir zone do not contribute to permeability.

In some examples, the frac mud is injected into a reservoir zone through perforation zone 304. The perforation zone 304 is determined using the example process 100. The frac mud is a specialized fluid used in hydraulic fracturing operations to create fractures in underground formations and facilitate the extraction of oil and gas. The reservoir zone has a high gas flow rate that meets production requirements. The reservoir zone is a high permeability zone with a high flow rate and a high wellhead pressure. The layers in the sand package of the reservoir zone are interconnected, and the measured pressure and fluid transferability are also within a predicted range. The perforation zone 304 is located within the specific zone isolated by the packer 238, and thus the perforation zone 304 is located at a depth corresponding to a depth of the injectable zone 306. The perforation zone 304 is recommended as the most likely best perforation zone 304 for the reservoir.

The process 100 of FIG. 1 can provide reservoir management and completion operation to identify the best perforation zone. In particular, the process 100 of FIG. 1 can identify a depth where the perforation can be effectively executed and a sand thickness required to achieve the targeted production rate. The sand thickness is the continuous thickness based on the Volume of Sand and Volume of Silt cutoffs. The thickness varies by geological setting. The process 100 of FIG. 1 can save the costs of running additional packers and perforating unnecessary intervals that may not be productive. The accuracy of the process 100 in FIG. 1 is exceeding 85% when compared to the expected results.

The present techniques can identify productive layers below the company's net pay and net reservoir petrophysical parameters threshold values. The present techniques can identify productive intervals and nonproductive intervals in a well. The present techniques can re-layer a massive clastic reservoir in a simulation model, which improves the field reservoir pressure history matching. Field reservoir pressure history matching is a process used in reservoir engineering to calibrate numerical simulation models with historical pressure data collected from the field. The field reservoir pressure history matching can adjust the parameters of the simulation model to accurately replicate the observed pressure behavior in the reservoir over time.

Furthermore, the present techniques can provide a precise depth for the most likely injectable and productive perforation zone and identify layers with non-movable fluid/water. The present techniques can expedite the completion stage and optimize the number of packers required for the perforation operation. The present techniques can likely select the best packages of sandstone reservoir for production and maximize drainage from the most productive layers/packages. The operational cost can be significantly reduced by optimizing the number of required perforations.

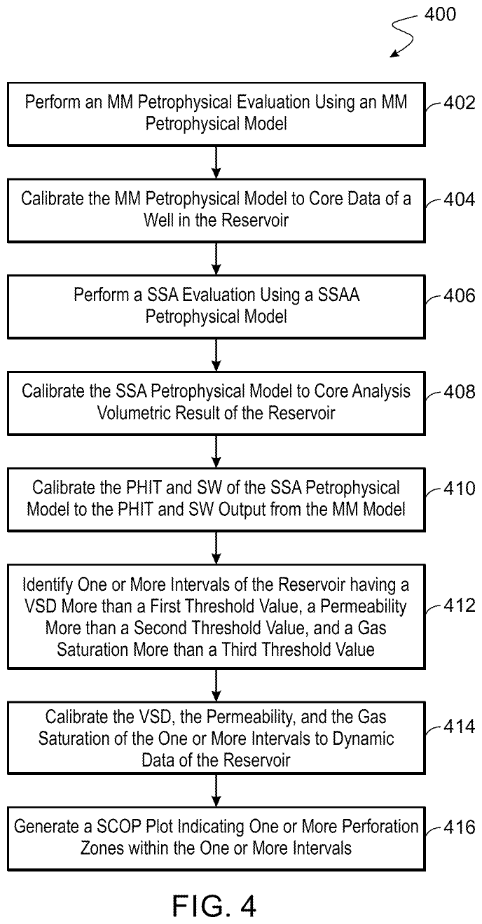

FIG. 4 illustrates a flow chart of a process 400 for identifying a perforation zone for the production of hydrocarbons, according to some implementations. The SCOP can identify the most likely effective perforation zone for hydrocarbon production. The process 400 is described as being performed by a computing device including one or more processors or a controller, such as controller 600 of FIG. 6. The process 400 shown in FIG. 4 can be modified or reconfigured to include additional, fewer, or different steps (even if not shown in FIG. 4), which can be performed in the order shown or in a different order.

At 402, the controller performs an MM petrophysical evaluation using an MM petrophysical model. In some implementations, the output of the MM petrophysical model is a plot as shown in FIG. 2A.

At 404, the controller calibrates the MM petrophysical model to core data of a well in the reservoir. Calibration of the MM petrophysical model to PHIT, permeability, and SW data in the core data can improve the accuracy and reliability of the MM petrophysical model's predictions and enhance understanding of the reservoir's lithology, mineralogy, and fluid distribution.

At 406, the controller performs a SSA evaluation using a SSA petrophysical model. In some implementations, the output of the SSA petrophysical model is a plot as shown in FIG. 2B.

At 408, the controller calibrates the SSA petrophysical model to core analysis volumetric result of the reservoir. The core analysis volumetric result can be obtained by deterministic description or AML XRD/XRF. Calibration of the SSA petrophysical model to core analysis volumetric results can improve the accuracy and reliability of the model's predictions regarding reservoir properties.

At 410, the controller calibrates the PHIT and SW of the SSA petrophysical model to the PHIT and SW output from the MM petrophysical model. The calibration involves adjusting the parameters and relationships within the SSA petrophysical model to match or align with the PHIT and SW values generated by the MM petrophysical model. During the calibration process, the parameters within the SSA petrophysical model related to PHIT and SW calculations are adjusted iteratively until the output values closely match the corresponding values from the MM petrophysical model. This adjustment may involve fine-tuning equations, coefficients, or assumptions within the SSA petrophysical model to better capture the lithological and fluid properties of the reservoir as represented by the MM petrophysical model.

At 412, the controller identifies one or more intervals of the reservoir having a VSD more than a first threshold value, a permeability more than a second threshold value, and a gas saturation more than a third threshold value. The output of the one or more intervals is a plot as shown in FIG. 2C.

At 414, the controller calibrates the VSD, the permeability, and the gas saturation of the one or more intervals to dynamic data of the reservoir. The calibration involves adjusting the parameters and relationships within the SSA petrophysical model to match or align with the MM petrophysical model, as well as the observed reservoir behavior and properties obtained from dynamic measurements and tests.

At 416, if the VSD, the permeability, and the gas saturation match the dynamic data at a degree more than a threshold value (e.g., 80% or more), the controller generates a SCOP plot (as shown in FIG. 3) indicating one or more perforation zones within the one or more intervals. If the VSD, the permeability, and the gas saturation match the dynamic data at a degree less than the threshold value (e.g., less than 80%), the controller reverts to 406 and iteratively performs 406-414 until the VSD, the permeability, and the gas saturation match the dynamic data at a degree more than the threshold value.

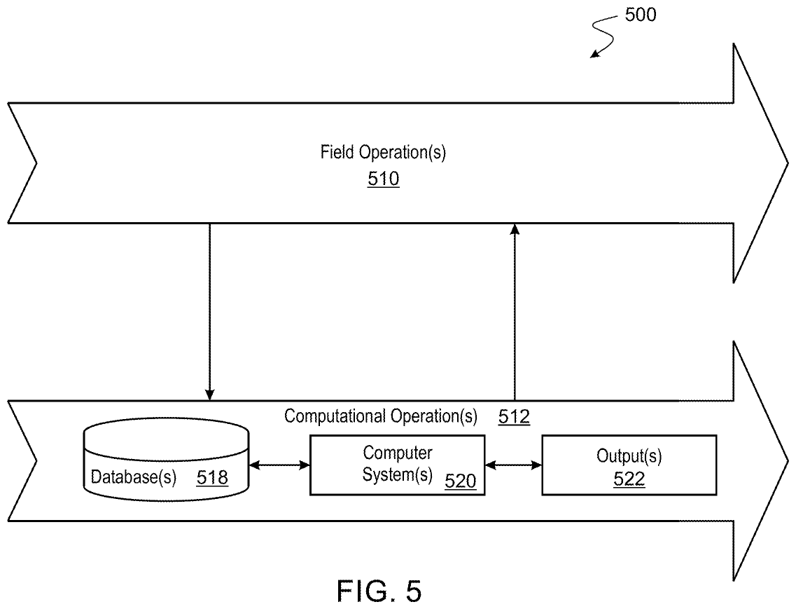

FIG. 5 illustrates hydrocarbon production operations 500 that include both one or more field operations 510 and one or more computational operations 512, which exchange information and control exploration for the production of hydrocarbons, according to some implementations. In some implementations, outputs of techniques of the present disclosure can be performed before, during, or in combination with the hydrocarbon production operations 500, specifically, for example, either as field operations 510 or computational operations 512, or both.

Examples of field operations 510 include forming/drilling a wellbore, hydraulic fracturing, producing through the wellbore, injecting fluids (such as water) through the wellbore, to name a few. In some implementations, methods of the present disclosure can trigger or control the field operations 510. For example, the methods of the present disclosure can generate data from hardware/software including sensors and physical data gathering equipment (e.g., seismic sensors, well logging tools, flow meters, and temperature and pressure sensors). The methods of the present disclosure can include transmitting the data from the hardware/software to the field operations 510 and responsively triggering the field operations 510 including, for example, generating plans and signals that provide feedback to and control physical components of the field operations 510. Alternatively or in addition, the field operations 510 can trigger the methods of the present disclosure. For example, implementing physical components (including, for example, hardware, such as sensors) deployed in the field operations 510 can generate plans and signals that can be provided as input or feedback (or both) to the methods of the present disclosure.

Examples of computational operations 512 include one or more computer systems 520 that include one or more processors and computer-readable media (e.g., non-transitory computer-readable media) operatively coupled to the one or more processors to execute computer operations to perform the methods of the present disclosure. The computational operations 512 can be implemented using one or more databases 518, which store data received from the field operations 510 and/or generated internally within the computational operations 512 (e.g., by implementing the methods of the present disclosure) or both. For example, the one or more computer systems 520 process inputs from the field operations 510 to assess conditions in the physical world, the outputs of which are stored in the databases 518. For example, seismic sensors of the field operations 510 can be used to perform a seismic survey to map subterranean features, such as facies and faults. In performing a seismic survey, seismic sources (e.g., seismic vibrators or explosions) generate seismic waves that propagate in the earth and seismic receivers (e.g., geophones) measure reflections generated as the seismic waves interact with boundaries between layers of a subsurface formation. The source and received signals are provided to the computational operations 512 where they are stored in the databases 518 and analyzed by the one or more computer systems 520.

In some implementations, one or more outputs 522 generated by the one or more computer systems 520 can be provided as feedback/input to the field operations 510 (either as direct input or stored in the databases 518). The field operations 510 can use the feedback/input to control physical components used to perform the field operations 510 in the real world.

For example, the computational operations 512 can process the seismic data to generate three-dimensional (3D) maps of the subsurface formation. The computational operations 512 can use these 3D maps to provide plans for locating and drilling exploratory wells. In some operations, the exploratory wells are drilled using logging-while-drilling (LWD) techniques which incorporate logging tools into the drill string. LWD techniques can enable the computational operations 512 to process new information about the formation and control the drilling to adjust to the observed conditions in real time.

The one or more computer systems 520 can update the 3D maps of the subsurface formation as information from one exploration well is received and the computational operations 512 can adjust the location of the next exploration well based on the updated 3D maps. Similarly, the data received from production operations can be used by the computational operations 512 to control components of the production operations. For example, production well and pipeline data can be analyzed to predict slugging in pipelines leading to a refinery and the computational operations 512 can control machine-operated valves upstream of the refinery to reduce the likelihood of plant disruptions that run the risk of taking the plant offline.

In some implementations of the computational operations 512, customized user interfaces can present intermediate or final results of the above-described processes to a user. Information can be presented in one or more textual, tabular, or graphical formats, such as through a dashboard. The information can be presented at one or more on-site locations (such as at an oil well or other facility), on the Internet (such as on a webpage), on a mobile application (or app), or at a central processing facility.

The presented information can include feedback, such as changes in parameters or processing inputs, that the user can select to improve a production environment, such as in the exploration, production, and/or testing of petrochemical processes or facilities. For example, the feedback can include parameters that, when selected by the user, can cause a change to, or an improvement in, drilling parameters (including drill bit speed and direction) or overall production of a gas or oil well. The feedback, when implemented by the user, can improve the speed and accuracy of calculations, streamline processes, improve models, and solve problems related to efficiency, performance, safety, reliability, costs, downtime, and the need for human interaction.

In some implementations, the feedback can be implemented in real-time, such as to provide an immediate or near-immediate change in operations or in a model. The term real-time (or similar terms as understood by one of ordinary skill in the art) means that an action and a response are temporally proximate such that an individual perceives the action and the response occurring substantially simultaneously. For example, the time difference for a response to display (or for initiation of a display) of data following the individual's action to access the data can be less than 1 millisecond (ms), less than 1 second (s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

Events can include readings or measurements captured by downhole equipment such as sensors, pumps, bottom-hole assemblies, or other equipment. The readings or measurements can be analyzed at the surface, such as by using applications that can include modeling applications and machine learning. The analysis can be used to generate changes to settings of downhole equipment, such as drilling equipment. In some implementations, values of parameters or other variables that are determined can be used automatically (such as through using rules) to implement changes in oil or gas well exploration, production/drilling, or testing. For example, outputs of the present disclosure can be used as inputs to other equipment and/or systems at a facility. This can be especially useful for systems or various pieces of equipment that are located several meters or several miles apart, or are located in different countries or other jurisdictions.

FIG. 6 is a schematic illustration of an example controller 600 (or control system) that enables a SCOP to identify a perforation zone for the production of hydrocarbons, according to some implementations. For example, the controller 600 may be operable according to the process 100 of FIG. 1 and process 400 of FIG. 4. The controller 600 is intended to include various forms of digital computers, such as printed circuit boards (PCB), processors, digital circuitry, or otherwise parts of a system for supply chain alert management. Additionally, the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The controller 600 includes a processor 610, a memory 620, a storage device 630, and an input/output interface 640 communicatively coupled with input/output devices 660 (for example, displays, keyboards, measurement devices, sensors, valves, pumps). Each of the components 610, 620, 630, and 640 are interconnected using a system bus 650. The processor 610 is capable of processing instructions for execution within the controller 600. The processor may be designed using any of a number of architectures. For example, the processor 610 may be a CISC (Complex Instruction Set Computers) processor, a RISC (Reduced Instruction Set Computer) processor, or a MISC (Minimal Instruction Set Computer) processor.

In one implementation, the processor 610 is a single-threaded processor. In another implementation, the processor 610 is a multi-threaded processor. The processor 610 is capable of processing instructions stored in the memory 620 or on the storage device 630 to display graphical information for a user interface on the input/output interface 640.

The memory 620 stores information within the controller 600. In one implementation, the memory 620 is a computer-readable medium. In one implementation, the memory 620 is a volatile memory unit. In another implementation, the memory 620 is a non-volatile memory unit.

The storage device 630 is capable of providing mass storage for the controller 600. In one implementation, the storage device 630 is a computer-readable medium. In various different implementations, the storage device 630 may be a floppy disk device, a hard disk device, an optical disk device, or a tape device.

The input/output interface 640 provides input/output operations for the controller 600. In one implementation, the input/output devices 660 include a keyboard and/or pointing device. In another implementation, the input/output devices 660 include a display unit for displaying graphical user interfaces.

There can be any number of controllers 600 associated with, or external to, a computer system containing controller 600, with each controller 600 communicating over a network. Further, the terms “client,” “user,” and other appropriate terminology can be used interchangeably, as appropriate, without departing from the scope of the present disclosure. Moreover, the present disclosure contemplates that many users can use one controller 600 and one user can use multiple controllers 600.

### EMBODIMENTS/EXAMPLES

According to some non-limiting implementations or examples, provided is a computer-implemented method, including: performing, by one or more processors, a Multimineral (MM) petrophysical evaluation using a MM petrophysical model; calibrating, by the one or more processors, the MM petrophysical model to core data of a well in the reservoir; performing, by the one or more processors, a shaly-sand-analysis (SSA) evaluation using a SSA petrophysical model; calibrating, by the one or more processors, the SSA petrophysical model to core analysis volumetric result of the reservoir; calibrating, by the one or more processors, porosity (PHIT) and water saturation (SW) of the SSA petrophysical model to PHIT and SW output from the MM petrophysical model; identifying, by the one or more processors, one or more intervals of the reservoir having a volume of sand (VSD) more than a first threshold value, a permeability more than a second threshold value, and a gas saturation more than a third threshold value; calibrating, by the one or more processors, the VSD, the permeability, and the gas saturation of the one or more intervals to dynamic data of the reservoir; and generating, by the one or more processors, a strategic completion optimization planner (SCOP) plot indicating one or more perforation zones within the one or more intervals.

According to some non-limiting implementations or examples, provided is a non-transitory, computer readable, storage medium that stores instructions that, when executed by at least one processor, cause the at least one processor to perform operations including: performing a Multimineral (MM) petrophysical evaluation using a MM petrophysical model; calibrating the MM petrophysical model to core data of a well in a reservoir; performing a shaly-sand-analysis (SSA) evaluation using a SSA petrophysical model; calibrating the SSA petrophysical model to core analysis volumetric result of the reservoir; calibrating porosity (PHIT) and water saturation (SW) of the SSA petrophysical model to PHIT and SW output from the MM petrophysical model; identifying one or more intervals of the reservoir having a volume of sand (VSD) more than a first threshold value, a permeability more than a second threshold value, and a gas saturation more than a third threshold value; calibrating the VSD, the permeability, and the gas saturation of the one or more intervals to dynamic data of the reservoir; and generating a strategic completion optimization planner (SCOP) plot indicating one or more perforation zones within the one or more intervals.

According to some non-limiting implementations or examples, provided is a system, including: one or more memory modules; one or more hardware processors communicably coupled to the one or more memory modules, the one or more hardware processors configured to execute instructions stored on the one or more memory models to perform operations comprising: performing a Multimineral (MM) petrophysical evaluation using a MM petrophysical model; calibrating the MM petrophysical model to core data of a well in a reservoir; performing a shaly-sand-analysis (SSA) evaluation using a SSA petrophysical model; calibrating the SSA petrophysical model to core analysis volumetric result of the reservoir; calibrating porosity (PHIT) and water saturation (SW) of the SSA petrophysical model to PHIT and SW output from the MM petrophysical model; identifying one or more intervals of the reservoir having a volume of sand (VSD) more than a first threshold value, a permeability more than a second threshold value, and a gas saturation more than a third threshold value; calibrating the VSD, the permeability, and the gas saturation of the one or more intervals to dynamic data of the reservoir; and generating a strategic completion optimization planner (SCOP) plot indicating one or more perforation zones within the one or more intervals.

Further non-limiting aspects or examples are set forth in the following numbered examples:

Example 1: A computer-implemented method for identifying a perforation zone for a reservoir, including: performing, by one or more processors, a Multimineral (MM) petrophysical evaluation using a MM petrophysical model; calibrating, by the one or more processors, the MM petrophysical model to core data of a well in the reservoir; performing, by the one or more processors, a shaly-sand-analysis (SSA) evaluation using a SSA petrophysical model; calibrating, by the one or more processors, the SSA petrophysical model to core analysis volumetric result of the reservoir; calibrating, by the one or more processors, porosity (PHIT) and water saturation (SW) of the SSA petrophysical model to PHIT and SW output from the MM petrophysical model; identifying, by the one or more processors, one or more intervals of the reservoir having a volume of sand (VSD) more than a first threshold value, a permeability more than a second threshold value, and a gas saturation more than a third threshold value; calibrating, by the one or more processors, the VSD, the permeability, and the gas saturation of the one or more intervals to dynamic data of the reservoir; and generating, by the one or more processors, a strategic completion optimization planner (SCOP) plot indicating one or more perforation zones within the one or more intervals.

Example 2: The computer-implemented method of Example 1, wherein generating the SCOP plot indicating the one or more perforation zones is performed in response to the VSD, the permeability, and the gas saturation matching the dynamic data at a degree more than a fourth threshold value.

Example 3: The computer-implemented method of Example 1 or 2, wherein the dynamic data comprises measurements from a Formation Testing with Sampling (FTS) tool and perforation results.

Example 4: The computer-implemented method of any one of previous Examples, further including: generating a gross sand flag in response to the VSD of the one or more intervals being more than the first threshold value; generating a permeable layer flag in response to the permeability of the one or more intervals being more than the second threshold value; and generating a hydrocarbon flag in response to the gas saturation of the one or more intervals being more than the third threshold value.

Example 5: The computer-implemented method of any one of previous Examples, further including: obtaining the core analysis volumetric result by deterministic description or advanced mud logging (AML) X-Ray Diffraction (XRD)/X-Ray Fluorescence (XRF).

Example 6: The computer-implemented method of any one of previous Examples, wherein the core data comprises PHIT, a permeability, and SW data of the well.

Example 7: The computer-implemented method of any one of previous Examples, wherein the SCOP plot comprises a perforation depth and a perforation thickness of each perforation zone.

Example 8: A non-transitory, computer readable, storage medium that stores instructions that, when executed by at least one processor, cause the at least one processor to perform operations including: performing a Multimineral (MM) petrophysical evaluation using a MM petrophysical model; calibrating the MM petrophysical model to core data of a well in a reservoir; performing a shaly-sand-analysis (SSA) evaluation using a SSA petrophysical model; calibrating the SSA petrophysical model to core analysis volumetric result of the reservoir; calibrating porosity (PHIT) and water saturation (SW) of the SSA petrophysical model to PHIT and SW output from the MM petrophysical model; identifying one or more intervals of the reservoir having a volume of sand (VSD) more than a first threshold value, a permeability more than a second threshold value, and a gas saturation more than a third threshold value; calibrating the VSD, the permeability, and the gas saturation of the one or more intervals to dynamic data of the reservoir; and generating a strategic completion optimization planner (SCOP) plot indicating one or more perforation zones within the one or more intervals.

Example 9: The medium of Example 8, generating the SCOP plot indicating the one or more perforation zones is performed in response to the VSD, the permeability, and the gas saturation matching the dynamic data at a degree more than a fourth threshold value.

Example 10: The medium of Example 8 or 9, wherein the dynamic data comprises measurements from a Formation Testing with Sampling (FTS) tool and perforation results.

Example 11: The medium of any one of Examples 8-10, the operations further including: generating a gross sand flag in response to the VSD of the one or more intervals being more than the first threshold value; generating a permeable layer flag in response to the permeability of the one or more intervals being more than the second threshold value; and generating a hydrocarbon flag in response to the gas saturation of the one or more intervals being more than the third threshold value.

Example 12: The medium of any one of Examples 8-11, the operations further including: obtaining the core analysis volumetric result by deterministic description or advanced mud logging (AML) X-Ray Diffraction (XRD)/X-Ray Fluorescence (XRF).

Example 13: The medium of any one of Examples 8-12, wherein the core data comprises PHIT, a permeability, and SW data of the well.

Example 14: The medium of any one of Examples 8-13, wherein the SCOP plot comprises a perforation depth and a perforation thickness of each perforation zone.

Example 15: A computer-implemented system, including: one or more memory modules; one or more hardware processors communicably coupled to the one or more memory modules, the one or more hardware processors configured to execute instructions stored on the one or more memory models to perform operations including: one or more memory modules; one or more hardware processors communicably coupled to the one or more memory modules, the one or more hardware processors configured to execute instructions stored on the one or more memory models to perform operations comprising: performing a Multimineral (MM) petrophysical evaluation using a MM petrophysical model; calibrating the MM petrophysical model to core data of a well in a reservoir; performing a shaly-sand-analysis (SSA) evaluation using a SSA petrophysical model; calibrating the SSA petrophysical model to core analysis volumetric result of the reservoir; calibrating porosity (PHIT) and water saturation (SW) of the SSA petrophysical model to PHIT and SW output from the MM petrophysical model; identifying one or more intervals of the reservoir having a volume of sand (VSD) more than a first threshold value, a permeability more than a second threshold value, and a gas saturation more than a third threshold value; calibrating the VSD, the permeability, and the gas saturation of the one or more intervals to dynamic data of the reservoir; and generating a strategic completion optimization planner (SCOP) plot indicating one or more perforation zones within the one or more intervals.

Example 16: The system of Example 15, generating the SCOP plot indicating the one or more perforation zones is performed in response to the VSD, the permeability, and the gas saturation matching the dynamic data at a degree more than a fourth threshold value.

Example 17: The system of Example 15 or 16, wherein the dynamic data comprises measurements from a Formation Testing with Sampling (FTS) tool and perforation results.

Example 18: The system of any one of Examples 15-17, the operations further including generating a gross sand flag in response to the VSD of the one or more intervals being more than the first threshold value; generating a permeable layer flag in response to the permeability of the one or more intervals being more than the second threshold value; and generating a hydrocarbon flag in response to the gas saturation of the one or more intervals being more than the third threshold value.

Example 19: The system of any one of Examples 15-18, the operations further including obtaining the core analysis volumetric result by deterministic description or advanced mud logging (AML) X-Ray Diffraction (XRD)/X-Ray Fluorescence (XRF).

Example 20: The system of any one of Examples 15-19, wherein the core data includes PHIT, a permeability, and SW data of the well.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example, LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory. A computer can also include, or be operatively coupled to, one or more mass storage devices for storing data. In some implementations, a computer can receive data from, and transfer data to, the mass storage devices including, for example, magnetic, magneto optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer readable media can also include magneto optical disks and optical memory devices and technologies including, for example, digital video disc (DVD), CD ROM, DVD+/−R, DVD-RAM, DVD-ROM, HD-DVD, and BLU-RAY.

The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touchscreen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that is used by the user. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component, for example, as a data server, or that includes a middleware component, for example, an application server. Moreover, the computing system can include a front-end component, for example, a client computer having one or both of a graphical user interface or a Web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship. Cluster file systems can be any file system type accessible from multiple servers for read and update. Locking or consistency tracking may not be necessary since the locking of exchange file system can be done at application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Various components may be described as performing a task or tasks, for convenience in the description. Such descriptions should be interpreted as including the phrase “configured to.” Reciting a component that is configured to perform one or more tasks is expressly intended not to invoke 35 USC § 112(f) interpretation for that component.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

Particular implementations of the subject matter have been described. Other implementations are within the scope of the following claims. For example, the actions recited in the claims can be performed in a different order and still achieve desirable results. As one example, some processes depicted in the accompanying figures do not necessarily require the particular order shown, or sequential order, to achieve desirable results.

We claim:

1. A computer-implemented method for identifying a perforation zone for a reservoir, comprising:
performing, by one or more processors, a Multimineral (MM) petrophysical evaluation using a MM petrophysical model;
calibrating, by the one or more processors, the MM petrophysical model to core data of a well in the reservoir;
performing, by the one or more processors, a shaly-sand-analysis (SSA) evaluation using a SSA petrophysical model;
calibrating, by the one or more processors, the SSA petrophysical model to core analysis volumetric result of the reservoir;
calibrating, by the one or more processors, porosity (PHIT) and water saturation (SW) of the SSA petrophysical model to PHIT and SW output from the MM petrophysical model;
identifying, by the one or more processors, one or more intervals of the reservoir having a volume of sand (VSD) more than a first threshold value, a permeability more than a second threshold value, and a gas saturation more than a third threshold value;
calibrating, by the one or more processors, the VSD, the permeability, and the gas saturation of the one or more intervals to dynamic data of the reservoir; and
generating, by the one or more processors, a strategic completion optimization planner (SCOP) plot indicating one or more perforation zones within the one or more intervals.

2. The computer-implemented method of claim 1, wherein generating the SCOP plot indicating the one or more perforation zones is performed in response to the VSD, the permeability, and the gas saturation matching the dynamic data at a degree more than a fourth threshold value.

3. The computer-implemented method of claim 1, wherein the dynamic data comprises measurements from a Formation Testing with Sampling (FTS) tool and perforation results.

4. The computer-implemented method of claim 1, further comprising:
generating a gross sand flag in response to the VSD of the one or more intervals being more than the first threshold value;
generating a permeable layer flag in response to the permeability of the one or more intervals being more than the second threshold value; and
generating a hydrocarbon flag in response to the gas saturation of the one or more intervals being more than the third threshold value.

5. The computer-implemented method of claim 1, further comprising:
obtaining the core analysis volumetric result by deterministic description or advanced mud logging (AML) X-Ray Diffraction (XRD)/X-Ray Fluorescence (XRF).

6. The computer-implemented method of claim 1, wherein the core data comprises PHIT, a permeability, and SW data of the well.

7. The computer-implemented method of claim 1, wherein the SCOP plot comprises a perforation depth and a perforation thickness of each perforation zone.

8. A non-transitory, computer readable storage medium that stores instructions that, when executed by at least one processor, cause the at least one processor to perform operations comprising:
performing a Multimineral (MM) petrophysical evaluation using a MM petrophysical model;
calibrating the MM petrophysical model to core data of a well in a reservoir;
performing a shaly-sand-analysis (SSA) evaluation using a SSA petrophysical model;
calibrating the SSA petrophysical model to core analysis volumetric result of the reservoir;
calibrating porosity (PHIT) and water saturation (SW) of the SSA petrophysical model to PHIT and SW output from the MM petrophysical model;
identifying one or more intervals of the reservoir having a volume of sand (VSD) more than a first threshold value, a permeability more than a second threshold value, and a gas saturation more than a third threshold value;
calibrating the VSD, the permeability, and the gas saturation of the one or more intervals to dynamic data of the reservoir; and
generating a strategic completion optimization planner (SCOP) plot indicating one or more perforation zones within the one or more intervals.

9. The non-transitory, computer readable storage medium of claim 8, generating the SCOP plot indicating the one or more perforation zones is performed in response to the VSD, the permeability, and the gas saturation matching the dynamic data at a degree more than a fourth threshold value.

10. The non-transitory, computer readable storage medium of claim 8, wherein the dynamic data comprises measurements from a Formation Testing with Sampling (FTS) tool and perforation results.

11. The non-transitory, computer readable storage medium of claim 8, the operations further comprising:
generating a gross sand flag in response to the VSD of the one or more intervals being more than the first threshold value;
generating a permeable layer flag in response to the permeability of the one or more intervals being more than the second threshold value; and
generating a hydrocarbon flag in response to the gas saturation of the one or more intervals being more than the third threshold value.

12. The non-transitory, computer readable storage medium of claim 8, the operations further comprising:
obtaining the core analysis volumetric result by deterministic description or advanced mud logging (AML) X-Ray Diffraction (XRD)/X-Ray Fluorescence (XRF).

13. The non-transitory, computer readable storage medium of claim 8, wherein the core data comprises PHIT, a permeability, and SW data of the well.

14. The non-transitory, computer readable storage medium of claim 8, wherein the SCOP plot comprises a perforation depth and a perforation thickness of each perforation zone.

15. A computer-implemented system, comprising:
one or more memory modules;
one or more hardware processors communicably coupled to the one or more memory modules, the one or more hardware processors configured to execute instructions stored on the one or more memory modules to perform operations comprising:
performing a Multimineral (MM) petrophysical evaluation using a MM petrophysical model;
calibrating the MM petrophysical model to core data of a well in a reservoir;
performing a shaly-sand-analysis (SSA) evaluation using a SSA petrophysical model;
calibrating the SSA petrophysical model to core analysis volumetric result of the reservoir;
calibrating porosity (PHIT) and water saturation (SW) of the SSA petrophysical model to PHIT and SW output from the MM petrophysical model;
identifying one or more intervals of the reservoir having a volume of sand (VSD) more than a first threshold value, a permeability more than a second threshold value, and a gas saturation more than a third threshold value;
calibrating the VSD, the permeability, and the gas saturation of the one or more intervals to dynamic data of the reservoir; and
generating a strategic completion optimization planner (SCOP) plot indicating one or more perforation zones within the one or more intervals.

16. The computer-implemented system of claim 15, generating the SCOP plot indicating the one or more perforation zones is performed in response to the VSD, the permeability, and the gas saturation matching the dynamic data at a degree more than a fourth threshold value.

17. The computer-implemented system of claim 15, wherein the dynamic data comprises measurements from a Formation Testing with Sampling (FTS) tool and perforation results.

18. The computer-implemented system of claim 15, the operations further comprising:
generating a gross sand flag in response to the VSD of the one or more intervals being more than the first threshold value;
generating a permeable layer flag in response to the permeability of the one or more intervals being more than the second threshold value; and
generating a hydrocarbon flag in response to the gas saturation of the one or more intervals being more than the third threshold value.

19. The computer-implemented system of claim 15, the operations further comprising:
obtaining the core analysis volumetric result by deterministic description or advanced mud logging (AML) X-Ray Diffraction (XRD)/X-Ray Fluorescence (XRF).

20. The computer-implemented system of claim 15, wherein the core data comprises PHIT, a permeability, and SW data of the well.
